(19) United States
(12) Patent Application Publication
(10) Pub. No.: US 2022/0192916 A1
(43) Pub. Date: Jun. 23, 2022

(54) THERMAL VIBRATION FOAM ROLLER

(71) Applicant: Kalum LAMOTHE, Edmonton (CA)

(72) Inventor: Kalum LAMOTHE, Edmonton (CA)

(21) Appl. No.: 17/600,970

(22) PCT Filed: Apr. 3, 2020
(86) PCT No.: PCT/CA2020/050449

§ 371 Date: Oct. 1, 2021

Related U.S. Application Data
(60) Provisional application No. 62/829,805, filed on Apr. 5, 2019.

(51) Int. Cl. A61H 15/02; A61H 23/02

(52) U.S. Cl. CPC A61H 15/02; A61H 2015/0014; A61H 23/0254

(57) ABSTRACT

A cordless foam roller applies heat and vibration through a rotary motor to muscles, and operates using an internal rechargeable power source when in use. The cordless foam roller includes an elongated cylindrically-shaped body having a textured exterior surface, and defining an inner cavity for receiving and accommodating an inner console; the inner console defining an inner cavity housing one or more of a circuit board comprising a microcontroller and heat and vibration setting buttons; a battery; and a rotary motor; and having an outer surface supporting one or more heating elements thereon. The foam roller is used for musculoskeletal rehabilitative, therapeutic and stretching programs.

### CROSS-REFERENCE TO RELATED APPLICATION

This application claims priority of U.S. Provisional Patent Application Ser. No. 62/829,805, entitled “Thermal Vibration Foam Roller,” filed Apr. 5, 2019, and hereby incorporated by reference herein in its entirety (where permitted).

### FIELD OF THE INVENTION

The present invention relates generally to a cordless foam roller for musculoskeletal rehabilitative, therapeutic and stretching programs, and more particularly, to a foam roller for applying heat and vibration to muscles.

### BACKGROUND OF THE INVENTION

Fascia is a fibrous, soft connective tissue that permeates the human body, and acts as a web of tissue that surrounds all components and compartments of the body to maintain integrity, support, and protective structure. Foam rolling is a common form of self-myofascial release which is often used by athletes prior to a workout to improve flexibility or after a workout to reduce muscle soreness and promote quicker recovery. Foam rolling involves lying on the ground, with the foam roller placed between an athlete's body and ground. The foam roller is pressed into the muscles being treated, and the athlete moves his body back and forth, thereby rolling along the ground. In this manner, deep compression of self-myofascial release enables release of deep tissue tension or trigger points, and normal blood flow to the muscles, thereby facilitating healing, recovery, and normal function of the treated muscles.

Conventional foam rollers vary with respect to density, surface texture, shape, size, scope, and application. Density is a significant factor for the effectiveness of self-myofascial release. Soft rollers (e.g., polyethylene, ethyl vinyl acetate, polyurethane, and expanded polypropylene) tend to provide gentle massage but inadequate pressure, disintegrate prematurely, or lose elasticity and rigidity after repeated use. Hard rollers (e.g., polyvinyl chloride, ethylene vinyl acetate-polyvinyl chloride mix, ethylene vinyl acetate-polyolefin mix) provide a deeper, more intense massage, are more durable to maintain their form after repeated heavy use, but cause bruising and pain. Surface textures vary from smooth to provide even pressure across the entire length of the roller, to textured (e.g., having ridges, knobs, points, spikes) to provide targeted massage on tight, compact muscles. Foam rollers are typically cylindrically-shaped and available in various sizes. “Long” rollers of about 36 inches are stable for use on quadriceps, hamstrings, upper back, and shoulders. The larger the body surface to target, the longer the foam roller required. “Short” rollers of about 24 inches target smaller areas such as arms and calves, while even shorter rollers of about 4 to 12 inches are compact, portable, and useful in limited floor space. Foam rollers range in diameter between about 5 to 6 inches, or 3 to 4 inches for deeper, targeted massage.

However, there are limitations associated with such conventional foam rollers since gyms must supply a range of foam rollers having various dimensions to athletes, or athletes themselves must purchase more than one foam roller in order to target various specific muscles. Particular dimensions of foam rollers may restrict their portability or controllability. For example, long foam rollers are too lengthy to store and carry in a gym bag. Short foam rollers are not wide enough to target large muscles such as those of the back, causing the athlete to roll off such that discomfort or possibly injury may occur due to limited control of the body and the short foam roller.

Attempts have been made to enhance conventional foam rollers by adding vibration to yield various designs of vibrating foam rollers. Physiological benefits of vibration include, for example, deeper, more intense massage; greater loosening and lengthening of muscles to increase flexibility; increased circulation; reduction of muscle soreness and stiffness; and reduction of pain associated with myofascial release. A conventional foam roller has been modified to provide heat therapy to raise muscle temperature for warm up, recovery, and pain relief, but must be pre-heated in a microwave oven which is unlikely to be present or conveniently accessible in a gym. Further, the heat dissipates quickly during use, and is only of short duration.

Accordingly, there is a need in the art for a greatly improved foam roller for potential musculoskeletal rehabilitative, therapeutic and stretching programs.

### SUMMARY OF THE INVENTION

The present invention relates generally to a cordless foam roller for musculoskeletal rehabilitative, therapeutic and stretching programs, and more particularly, to a cordless foam roller for applying heat and vibration to muscles. It was surprisingly discovered that by utilizing the cordless foam roller and method of the present invention, one or more of the following benefits may be realized:

(1) The cordless foam roller of the present invention may be conveniently used to supply heat and vibration to different muscles for musculoskeletal rehabilitative, therapeutic and stretching programs. The heat elevates the temperature of the muscles for warm up, recovery, and pain relief. The vibration enables deeper, more intense massage; greater loosening and lengthening of muscles to increase flexibility; increased circulation; reduction of muscle soreness and stiffness; and reduction of pain associated with myofascial release. The user may choose to apply either heat or vibration alone, or simultaneously.

(2) The cordless foam roller is powered by a rechargeable battery, and thus can operate without power cords or cables attached to electrical outlets to provide mains power, allowing the foam roller to be self-contained (i.e., having an internal or built-in power supply) and having greater mobility during use.

(3) The body of the cordless foam roller is formed of a thermally conductive material, preferably a thermal silicone compound doped with silver nanoparticles for raising the thermal conductivity of the body. The presence of a specially designed thermally conductive material is to ensure that the material will not disintegrate upon exposure to heat and vibration, which is likely to occur with the foam used in conventional foam rollers.

(4) The presence of heating elements in the form of either resistive electrical wires or heating pads enables the cordless foam roller to heat itself, without requiring a microwave oven for heating as needed for a prior art device.

(5) The textured exterior surface of the cordless foam roller stimulates nerve endings more effectively and reaches deeper into the muscle's myofascial layers compared to a conventional soft foam roller having a smooth surface.

(6) The cordless foam roller has dimensions suitable for use with different targeted muscles, and is compact for storing in a gym bag or for use in a limited workout space, lightweight and portable for carrying to and from the gym, and useful for various large (e.g., back, shoulders) and small muscles (e.g., arms, calves) without risk of the user rolling off or lacking sufficient length to target a particular muscle.

Thus, broadly stated, in one aspect, the invention comprises a cordless foam roller comprising:
an elongated cylindrically-shaped body having a textured exterior surface, and defining an inner cavity for receiving and accommodating an inner console;
the inner console defining an inner cavity housing at least a circuit board comprising a microcontroller and heat and vibration setting buttons; a battery; and a rotary motor; and having an outer surface supporting one or more heating elements thereon.

In the various embodiments, the cordless foam roller further comprises a first end and a second end capped by a first end cap and a second end cap. In the various embodiments, the textured exterior surface comprises a tread of chevrons, each chevron being disposed with a channel defined between adjacent chevrons.

In the various embodiments, the body is formed of a thermally conductive material. In the various embodiments, the thermally conductive material is selected from silicone, a thermoplastic vulcanizate, or a thermal silicone compound doped with silver nanoparticles.

In the various embodiments, the inner console comprises a pipe formed of a thermoplastic polymer. In the various embodiments, the thermoplastic polymer comprises acrylonitrile butadiene styrene.

In the various embodiments, the microcontroller, the heat and vibration setting buttons, the battery, the rotary motor, and the one or more heating elements are operably connected to the circuit board. In the various embodiments, the microcontroller is configured to control power distribution to the battery and the rotary motor. In the various embodiments, the battery comprises a lithium-ion battery.

In the various embodiments, the microcontroller is configured to transmit a signal to the rotary motor to operate at a programmed frequency selected by a user. In the various embodiments, the rotary motor is configured to spin at the programmed frequency along the longitudinal axis of the foam roller and to emit vibrations in a perpendicular direction relative to the longitudinal axis of the foam roller.

In the various embodiments, the microcontroller is configured to transmit a signal to the battery to furnish electrical current sufficient for programmed heat selected by a user. In the various embodiments, the electrical current runs through the one or more heating elements for emitting heat, the heat being transferred through the body of the foam roller to the user.

In the various embodiments, the amount of heat ranges from about 35° C. to about 55° C. In the various embodiments, the heat settings equate to temperatures of about 35° C., about 45° C., and about 55° C.

In the various embodiments, the cordless foam roller further comprises at least one temperature sensor for detecting the temperature within the inner console and transmitting a signal to the microcontroller to increase or decrease the flow of the electrical current to the one or more heating elements. In the various embodiments, the one or more heating elements comprises one or more heating pads mounted on an entirety or a portion of an outer surface of the inner console, and operably connected to the microcontroller. In the various embodiments, the one or more heating elements comprises electrical resistive wires arranged in U-shapes along an entirety or portion of an outer surface of the inner console, and operably connected to the microcontroller.

In the various embodiments, the microcontroller is configured to transmit a signal to activate a cooling fan to cool the temperature of the inner console. In the various embodiments, the microcontroller is configured to monitor and control supply voltage available from the battery, and shut down the battery.

In another aspect, the invention comprises a method of facilitating a musculoskeletal rehabilitative, therapeutic or stretching program for a user, comprising the steps of:
providing a cordless foam roller comprising an elongated cylindrically-shaped body having a textured exterior surface, and defining an inner cavity for receiving and accommodating an inner console; the inner console defining an inner cavity housing at least a circuit board comprising a microcontroller and heat and vibration setting buttons; a battery; and a rotary motor; and having an outer surface supporting one or more heating elements thereon;
configuring the microcontroller to transmit a signal to the rotary motor to operate at a programmed frequency selected by the user, thereby spinning the rotary motor to emit vibration along the cordless foam roller; and
configuring the microcontroller to transmit a signal to the battery to furnish electrical current sufficient for the programmed heat selected by the user, the electrical current running through the one or more heating elements to emit heat transferable through the body of the cordless foam roller to the user.

In the various embodiments, the method further comprises the step of providing one or more sensors for detecting the temperature of the inner console. In the various embodiments, the method further comprises the step of adjusting the temperature of the inner console by increasing or decreasing the flow of the electrical current to the one or more heating elements.

Additional aspects and advantages of the present invention will be apparent in view of the description, which follows. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The invention will now be described by way of an exemplary embodiment with reference to the accompanying simplified, diagrammatic, not-to-scale drawings. In the drawings:

FIG. 1 is a side view of a first embodiment of a cordless foam roller of the present invention.

FIG. 2 is an exploded view of the embodiment of FIG. 1.

FIG. 3 is a side view of a second embodiment of a cordless foam roller of the present invention.

FIG. 4 is a rear view of the embodiment of FIG. 3.

FIG. 5 is an exploded view of the embodiment of FIG. 3.

FIG. 6 is an exploded view of the embodiment of FIG. 3.

### DETAILED DESCRIPTION OF PREFERRED EMBODIMENTS

Before the present invention is described in further detail, it is to be understood that the invention is not limited to the particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, a limited number of the exemplary methods and materials are described herein.

It must be noted that as used herein and in the appended claims, the singular forms “a,” “an,” and “the” include plural referents unless the context clearly dictates otherwise.

The present invention relates generally to a cordless foam roller which is configured to apply heat and vibration to muscles, and operates using an internal rechargeable power source when in use. As used herein, the term “cordless” means that the foam roller is powered by a battery, preferably a rechargeable battery, and can operate without a power cord, cable, or wires attached to an electrical outlet to provide mains power.

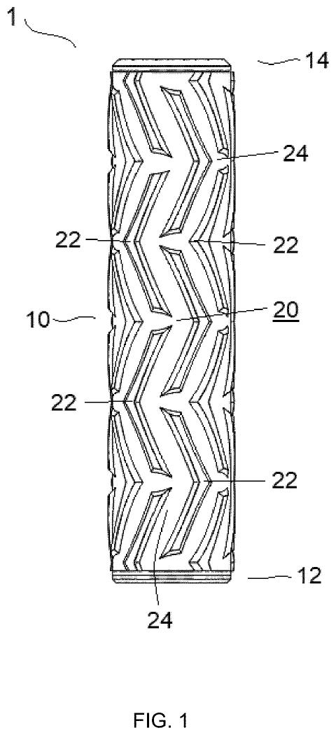

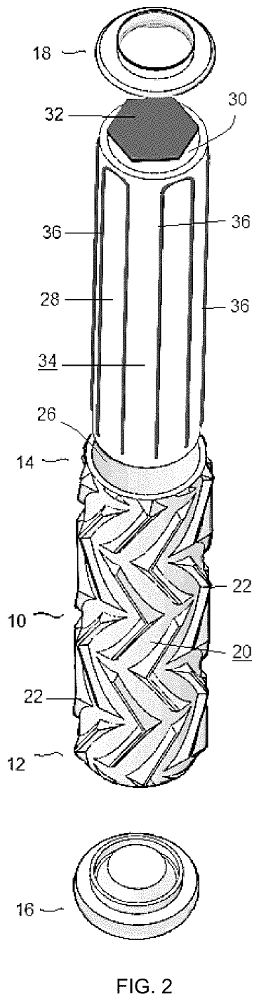

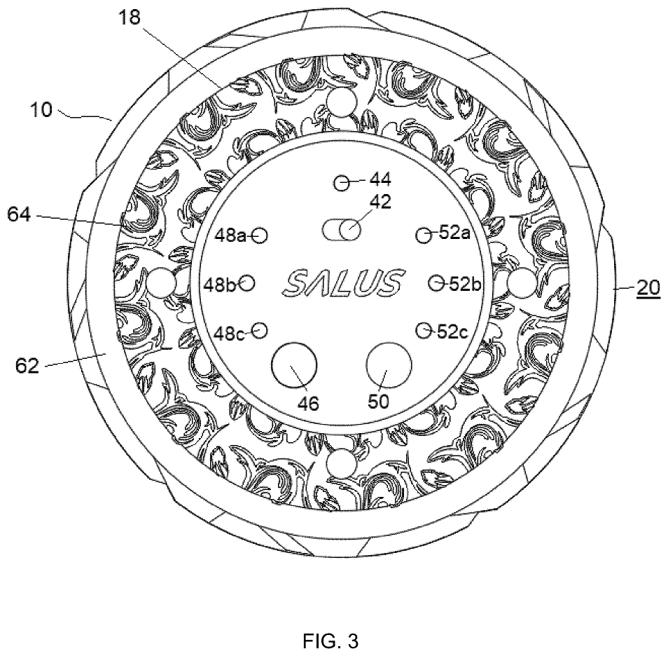

The invention will now be described having reference to the accompanying figures. FIGS. 1-2 show a first embodiment of a cordless foam roller of the present invention which is configured to apply heat through electrical resistive wires and vibration through a rotary motor to muscles, and operates using an internal rechargeable power source when in use. FIGS. 3-6 show a second embodiment of a cordless foam roller of the present invention which is configured to apply heat through one or more heating pads and vibration through a rotary motor to muscles, and operates using an internal rechargeable power source when in use. Since both embodiments of the foam roller are powered by a rechargeable battery, the foam rollers can operate without power cords or cables attached to electrical outlets to provide mains power, allowing the foam rollers to be self-contained (i.e., having an internal or built-in power supply) and having greater mobility during use. Both embodiments of the foam roller can also be used in the manner in which a typical foam roller is commonly employed for musculoskeletal rehabilitative, therapeutic and stretching programs.

In the various embodiments, the cordless foam roller (1) is shown generally in the Figures to include an elongated body (10) having a first end (12) and a second end (14) which are capped by a first end cap (16) and a second end cap (18), respectively.

The body (10) is substantially cylindrically-shaped having an exterior surface (20) which is textured along at least a portion or the entirety of the body (10). In the various embodiments, the exterior surface (20) is textured along the entirety of the body (10) along its length. In the various embodiments, the exterior surface (20) comprises a textured pattern or tread of chevrons (22), or V-shapes and inverted V-shapes imprinted thereon. It is contemplated that the number (density), size, and positioning of the chevrons (22) for the various embodiments of the cordless foam roller (1) may vary. In the various embodiments, the chevrons (22) are arranged as closely together as it practicable to maximize the contacting surface. In the various embodiments, each chevron (22) is disposed to almost abut an adjacent chevron (22) to define a channel (24) between the chevrons (22). The purpose of having a textured exterior surface (20) is to stimulate nerve endings more effectively and reach deeper into the muscle's myofascial layers compared to a conventional soft foam roller having a smooth surface.

In the various embodiments, the body (10) is formed of a thermally conductive material. As used herein, the term “thermally conductive” refers to the ability of a material to conduct or transfer heat. Given two surfaces on either side of the material with a temperature difference between them, the thermal conductivity is the heat energy transferred per unit time and per unit surface area, divided by the temperature difference. Suitable thermally conductive materials include, but are not limited to, silicone, thermoplastic vulcanizates (for example, Santoprene™), and the like. In the various embodiments, the body (10) is formed of a thermal silicone compound doped with silver nanoparticles for raising the thermal conductivity of the body (10). In the various embodiments, the body (10) is formed of a thermally conductive material exhibiting about 80% the conductivity of copper, and having a shore hardness of about 0.035. The purpose of forming the body (10) of a thermally conductive material is to ensure that the material will not disintegrate upon exposure to heat and vibration, which is likely to occur with the foam used in conventional foam rollers.

In the various embodiments, the body (10) is hollow to define an inner cavity (26) for receiving and accommodating an inner console (28). The inner console (28) has a length substantially the same or similar to the length of the body (10), and a diameter which is smaller than the diameter of the body (10) such that the inner console (28) is positioned telescopically within the body (10) when assembled. In the various embodiments, the body (10) preferably has a length of about 18.5 inches and a diameter of about 4.5 inches. In the various embodiments, the inner console (28) has a length of about 18 inches and a diameter of about 3 inches.

In the various embodiments, the inner console (28) comprises a pipe. The pipe may be formed of a thermoplastic polymer. In the various embodiments, the thermoplastic polymer comprises acrylonitrile butadiene styrene (commonly abbreviated as “ABS”) to confer impact resistance, toughness, and heat resistance.

In the various embodiments, the inner console (28) is hollow and defines an inner cavity (30) housing one or more of a circuit board (32) comprising a microcontroller, one or more function buttons associated with selected applications and corresponding indicators that indicate the functions associated with the function buttons, a battery (38), a rotary motor (40), and a cooling fan (not shown).

In the various embodiments, the one or more function buttons associated with a selected application may include touch or slider buttons having corresponding indicators displayed that indicate the function of the function buttons. In the various embodiments, the indicators are in the form of lights including, but not limited to, light-emitting diodes (“LEDs”). In the various embodiments, the function buttons and indicators may include, but are not limited to, an ON/OFF switch (42) and a corresponding ON/OFF indicator light (44); a heat setting button (46) and one or more corresponding heat setting indicator lights (48a, 48b, 48c); and a vibration setting button (50) and one or more corresponding vibration setting indicator lights (52a, 52b, 52c) (FIG. 3).

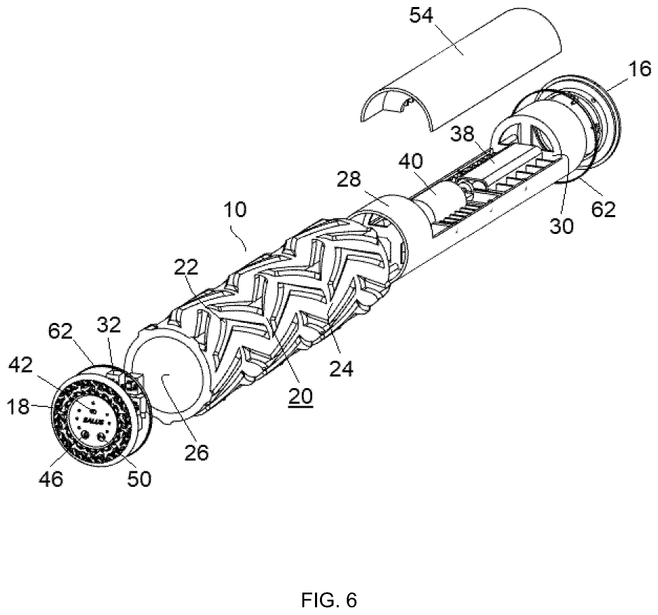

Any number of heat and vibration settings may be used. In the various embodiments, one heat setting indicator light (48a) indicates a temperature setting of 35° C.; one heat setting indicator light (48b) indicates a temperature setting of 45° C.; and one heat setting indicator light (48c) indicates a temperature setting of 55° C. In the various embodiments, one vibration setting indicator light (52a) indicates a speed setting of “low;” one vibration setting indicator light (52b) indicates a speed setting of “medium;” and one vibration setting indicator light (52c) indicates a speed setting of “high.” The low, medium, and high settings are determined by the desired range of frequency and voltage supplied to the rotary motor (40) as will be further described. In the various embodiments, a cover (54) is removably attached to cover various components of the inner console (28) such as, for example, the battery (38) and rotary motor (40) (FIG. 6).

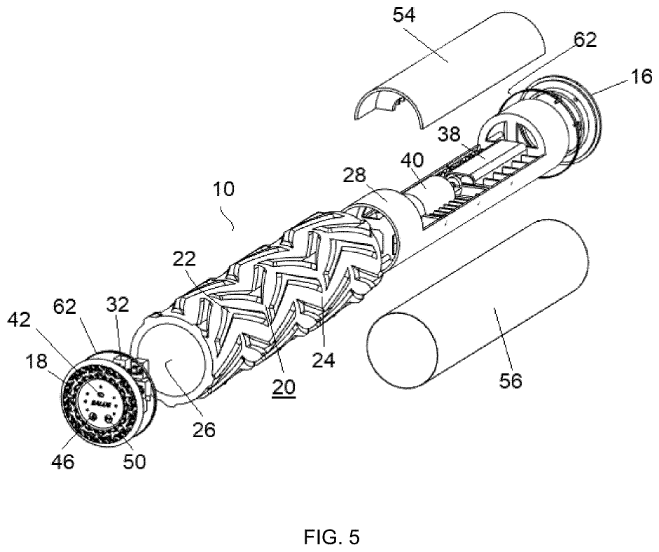

In the various embodiments, the inner console (28) has an outer surface (34) for supporting heating elements. In the various embodiments, the heating elements are in the form of either one or more electrical resistive wires (36) (FIG. 2), or one or more heating pads (56) (FIG. 5).

In the various embodiments, the circuit board (32) comprises a printed circuit board formed of a non-conductive material with conductive lines printed or etched. The microcontroller, function buttons and corresponding indicators, battery (38), rotary motor (40), cooling fan, wires (36) and heating pads (56) are connected either directly or indirectly to the circuit board (32) and traces may connect particular components together to form a working circuit or assembly. Such components generally perform all the functions of the foam roller (1) including, for example, emitting heat using the wires (36) or heating pads (56), reducing heat using the cooling fan, and emitting vibration using the rotary motor (40), as further described.

In the various embodiments, the microcontroller is mounted on the circuit board (32) and comprises a computer including at least one processor and at least one memory coupled to the processor and storing computer executable instructions which, when executed by the processor, perform particular operations to ensure proper functioning of the cordless foam roller (1). The microcontroller controls the power distribution to the battery (38), rotary motor (40), and cooling fan.

In the various embodiments, the battery (38) is connected to the circuit board (32). In the various embodiments, the battery (38) comprises a lithium-ion battery. As used herein, the term “lithium-ion battery” refers to a rechargeable battery in which lithium ions move from the negative electrode to the positive electrode during discharge and back when charging. The lithium-ion battery is preferred for having a substantially long life of about two the three years, high energy density, tiny memory effect, and low self-discharge.

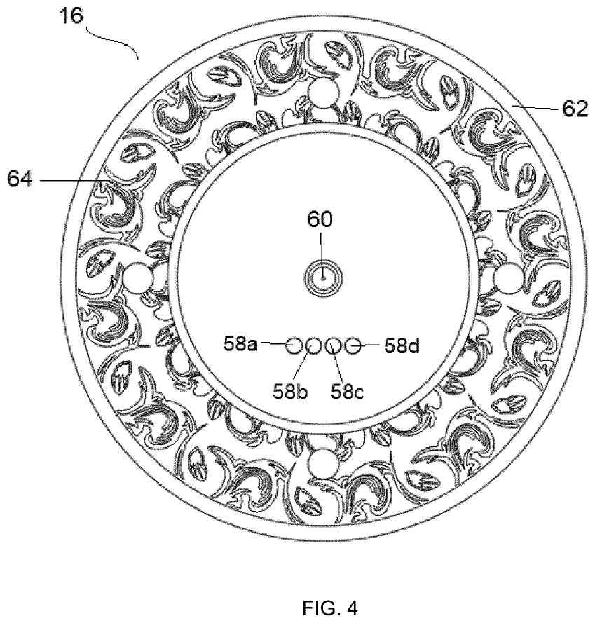

In the various embodiments, one or more LED indicator lights (58) indicate the battery power level (FIG. 4). In the various embodiments, a fully charged battery (38) may be indicated by having for example, all LED indicator lights (58a, 58b, 58c, 58d) in the “on” position. A battery having a power level between 75% to 99% may be indicated by having three LED indicator lights (58a, 58b, 58c) in the “on” position. A battery having a power level between 50% to 75% may be indicated by having two LED indicator lights (58a, 58b) in the “on” position. A battery having a power level between 25% to 50% may be indicated by having one LED indicator light (58a) in the “on” position. A battery having a power level between 0% to 25% may be indicated by having one LED indicator light (58a) “blinking” as an alert. In the various embodiments, a charging port (60) is provided for receiving a charging cable to recharge the battery (38). In the various embodiments, the charge port (60) may be provided within the first end cap (16) to facilitate recharging of the battery (FIG. 4).

In the various embodiments, the battery (38) powers the cooling fan which is activated to decrease or cool the temperature of the inner console (28) in the event that the temperature of the inner console (28) overheats beyond a predetermined maximum temperature. The cooling fan is deactivated once the temperature has been decreased to a preferred minimum temperature.

In the various embodiments, the temperature of the inner console (28) may be adjusted by increasing or decreasing the flow of the electrical current to the wires (36) or the heating pads (56).

In the various embodiments, the rotary motor (40) is connected to the circuit board (32) which drives the rotary motor (40) by varying the frequency and voltage supplied to the rotary motor (40). The frequency depends upon the setting selected by the user. In the various embodiments, at least one setting is provided of at least about 7.1 watts. In the various embodiments, there are three settings ranging from at least about 7.1 watts or greater. In the various embodiments, the rotary motor (40) is positioned within the inner cavity (30) at about the mid-length of the cordless foam roller (1). Upon receipt of signals from the microcontroller, the rotary motor (40) spins at the desired frequency along the longitudinal axis of the cordless foam roller (1) and emits vibration in a perpendicular direction relative to the longitudinal axis of the cordless foam roller (1), thereby transferring vibration along the cordless foam roller (1).

In the various embodiments, the electrical resistive wires (36) are mounted on the entirety or a portion of the outer surface (34) of the inner console (28) (FIG. 2). In the various embodiments, the wires (36) are arranged in “U”-shapes to run along the entirety or portion of the outer surface (34) of the inner console (28). The wires (36) connect to the circuit board (32) at the second end (18) of the body (10). The wires (36) provide a source of heat for the cordless foam roller (1). The amount of heat emitted by the wires (36) depends on the heat setting selected by the user. In the various embodiments, the amount of heat ranges from about 35° C. to about 55° C. In the various embodiments, 45° C. is the optimum temperature for therapy. In the various embodiments, three settings equating to temperatures of about 35° C., about 45° C., and about 55° C. are provided. Once a heat setting is selected, the microcontroller directs the flow of current through the wires (36) that act as resistors, emitting heat. The heat is transferred from the wires (36) through the thermally conductive body (10) of the cordless foam roller (1) to the user.

In the various embodiments, the heating pads (56) are mounted on the entirety or a portion of the outer surface (34) of the inner console (28) (FIG. 5). In the various embodiments, the heating pads (56) connect to the circuit board (32) at the second end (18) of the body (10). The heating pads (56) provide a source of heat for the cordless foam roller (1). The amount of heat emitted by the heating pads (56) depends on the heat setting selected by the user. In the various embodiments, the amount of heat ranges from about 35° C. to about 55° C. In the various embodiments, 45° C. is the optimum temperature for therapy. In the various embodiments, three settings equating to temperatures of about 35° C., about 45° C., and about 55° C. are provided. Once a heat setting is selected, the microcontroller directs the flow of current to the heating pads (56) that emit heat. The heat is transferred from the heating pads (56) through the thermally conductive body (10) of the cordless foam roller (1) to the user. In the various embodiments, the heating pads (56) are formed of a mesh of polyester filament and micro metal conductive fiber folded into a protective polyimide film. In various embodiments, the heating pads (56) use 24V of power at 3 amps when running on full power.

Without being bound by any theory, the heating pads (56) may be preferable over the resistive wires (36) due to having greater efficiency, effectiveness, and using less energy, thereby making the battery (38) last longer. The heating pads (56) may reach temperatures more quickly compared to the resistive wires (36). With use of the heating pads (56), the size of the battery (38) may be decreased, which in turn decreases the overall weight of the cordless foam roller (1).

Depending on the setting, the microcontroller monitors and controls the electrical current through the wires (36) or to the heating pads (56), and activates an emergency shut down if necessary. At least one temperature sensor is provided to detect the temperature. In the various embodiments, three temperature sensors are provided. In the various embodiments, in the event that the temperature exceeds the selected temperature, the sensor transmits a signal to the microcontroller to cease the flow of current to the wires (36) or to the heating pads (56), thereby halting the emission of heat. In the various embodiments, in the event that the temperature exceeds the selected temperature, the sensor transmits a signal to the microcontroller to cease the flow of current to the wires (36) or the heating pads (56), thereby halting the emission of heat. In addition, the microcontroller monitors and controls the supply voltage available from the battery (38), and shuts down the battery (38) in the event that the voltage becomes lower than a predetermined voltage, or the battery (38) overheats beyond a set maximum temperature.

In the various embodiments, the first end cap (16) is attachable over the first end (12) of the body (10) to seal the first end (12). In the various embodiments, the first end cap (16) is formed of a thermoplastic polymer. In the various embodiments, the thermoplastic polymer comprises ABS.

In the various embodiments, the second end cap (18) is attachable over the second end (14) of the body (10) to seal the second end (14). In the various embodiments, the second end cap (18) is attached over the circuit board (32). In the various embodiments, the second end cap (18) comprises a ring and a transparent lid mounted thereon. In the various embodiments, the ring is formed of a thermoplastic polymer. In the various embodiments, the thermoplastic polymer comprises ABS. In the various embodiments, the transparent lid is formed of plastic or glass. Having a transparent lid over the circuit board (32) allows the user to select the desired settings for heat and vibration.

In the various embodiments, the first and second end caps (16, 18) are securely attached to the cordless foam roller (1). Suitable attachment means include, but are not limited to, threads or adhesives such as glue, in order to prevent dislodgment of components within the inner cavity (30) of the cordless foam roller (1) during use. In the various embodiments, the first and second end caps (16, 18) define threads which mate with corresponding threads defined by the inner console (28) such that the first and second end caps (16, 18) may be threaded or screwed into and press against the inner console (28).

In the various embodiments, the cordless foam roller (1) may be embellished with one or more LEDs in a ring-like form (60a, 60b) or embossed designs (62) to improve aesthetics of the cordless foam roller (1).

The cordless foam roller (1) may be formed by processes known in the art. The cordless foam roller (1) can be constructed from any material or combination of materials having suitable properties such as, for example, mechanical strength, ability to withstand repeated heavy use, and ease of machining. The cordless foam roller (1) may be formed of appropriate materials known to those skilled in the art to ensure that the cordless foam roller (1) is as lightweight and portable as possible for easy handling by the user. In the various embodiments, the weight of the cordless foam roller (1) may range from about 7 pounds to about 10 pounds. In general, the cordless foam roller (1) requires few components, making the cordless foam roller (1) amenable to rapid assembly and minimizing expense in manufacturing.

The dimensions of the cordless foam roller (1) are not essential to the invention and may be increased or decreased as may be required to satisfy any particular design objectives. In the various embodiments, the cordless foam roller (1) preferably has a length of about 18.5 inches and a diameter of about 4.5 inches. This preferred length enables the cordless foam roller (1) to be compact, portable, and useful for various muscles without risk of the user rolling off or lacking sufficient length to target a particular muscle. In addition, the preferred diameter allows the cordless foam roller (1) to be suitable for deeper, targeted massage. The dimensions of the cordless foam roller (1) are distinct from conventional foam rollers which are either “long” rollers of about 36 inches or “short” rollers of about 12 inches or about 24 inches, with diameters for both long and short foam rollers between 5 to 6 inches, or 3 to 4 inches.

The components of the cordless foam roller (1) are assembled in the following manner. In the various embodiments, the inner console (28) is cut to the desired length. In the various embodiments, the inner console (28) comprises acrylonitrile butadiene styrene pipe which is cut to a length of about 18 inches.

In the various embodiments, the inner console (28) is lined with the electrical resistive wires (36) which are positioned along the length of the exterior surface (34) of the inner console (28) (FIG. 2). An end portion of each wire (36) is made available to be tucked into the inner console (28) during injection molding. The wires (36) are fixedly attached to the exterior surface (34) of the inner console (28) using suitable attachment means including, but not limited to, adhesives such as glue, in order to prevent the wires (36) from being dislodged from the desired position during injection molding.

In the various embodiments, the inner console (28) is lined with one or more heating pads (56) which are positioned along the length of the exterior surface (34) or a portion thereof of the inner console (28) (FIG. 5). In the various embodiments, the heating pads (56) are held in place by being sandwiched or pressed between the inner console (28) and the body (10) of the cordless foam roller (1). In the various embodiments, the heating pads (56) are attached to the exterior surface (34) of the console (28) using suitable attachment means including, but not limited to, adhesives such as glue, in order to prevent the heating pads (56) from being dislodged from the desired position, and connected together in series attaching to the circuit board (32).

Thermally conductive material forming the exterior surface (20) of the body (10) is injection molded around the inner console (28). The circuit board (32) comprising one or more of the microcontroller and the function buttons, battery (38), rotary motor (40), and cooling fan are then inserted into the inner cavity (30) of the inner console (28). In the various embodiments, the end portions of the wires (36) are attached and soldered to the circuit board (32), as are the wires for the battery (38), rotary motor (40), and cooling fan. The first and second end caps (16, 18) are secured for example, by threads or adhesive, to the respective first and second ends (12, 14) of the tubular member (28), locking the inner console (28) in place and securing all components together.

In the various embodiments, in operation, the user slides the ON/OFF switch (42) from “OFF” to “ON” which triggers the indicator light (44) showing that the cordless foam roller (1) is “ON” ready for use (FIG. 3). In various embodiments, the user presses the heat setting button (46) to select the desired temperature as indicated by the heat setting indicator lights (48a, 48b, 48c); for example, pressing the heat setting button (46) once selects 35° C. as indicated by the first heat setting indicator light (48a); pressing twice selects 45° C. as indicated by the second heat setting indicator light (48b); and pressing thrice selects 55° C. as indicated by the third heat setting indicator light (48c). In various embodiments, the user presses the vibration setting button (50) to select the desired vibration as indicated by the vibration setting indicator lights (52a, 52b, 52c); for example, pressing the vibration setting button (50) once selects “low” vibration as indicated by the first vibration setting indicator light (52a); pressing twice selects “medium” vibration as indicated by the second vibration setting indicator light (52b); and pressing thrice selects “high” vibration as indicated by the third vibration setting indicator light (52c).

The microcontroller transmits a signal to the rotary motor (40) to operate at the programmed frequency selected by the user. The rotary motor (40) spins along the longitudinal axis of the cordless foam roller (1) and emits vibration in a perpendicular direction relative to the longitudinal axis of the cordless foam roller (1), thereby transferring vibration along the cordless foam roller (1). The microcontroller transmits a signal to the battery (38) to furnish electrical current sufficient for the programmed heat selected by the user. In the various embodiments, the electrical current run through the wires (36) or the heating pads (56) which subsequently emit heat. The heat is transferred through the body (10) of the cordless foam roller (1) to the user.

The temperature sensors detect the temperature of the inner console (28) to ensure that the desired heat is being emitted in accordance with the user's selected heat setting, and that the inner console (28) does not overheat. The temperature sensors transmit signals to the microcontroller which monitors the temperature over time. In the various embodiments, if adjustment of the temperature is required, the microcontroller transmits a signal to activate the cooling fan. Operation of the cooling fan decreases the temperature. Once the temperature has been sufficiently cooled as detected by the temperature sensors, the microcontroller transmits a signal to deactivate the cooling fan. In the various embodiments, if adjustment of the temperature is required, the microcontroller transmits a signal to stop the transmission of electrical current to the wires (36) or the heating pads (56).

The cordless foam roller (1) of the present invention may be used in a variety of situations and in various ways. The cordless foam roller (1) may be conveniently used to supply heat and vibration to different muscles for musculoskeletal rehabilitative, therapeutic and stretching programs. The heat elevates the temperature of the muscles for warm up, recovery, and pain relief. The vibration enables deeper, more intense massage; greater loosening and lengthening of muscles to increase flexibility; increased circulation; reduction of muscle soreness and stiffness; and reduction of pain associated with myofascial release. The user may choose to apply either heat or vibration alone, or simultaneously. Preferably, heat and vibration are applied simultaneously for the user to experience the benefits of both treatments. The presence of resistive electrical wires (36) or heating pads (56) enables the cordless foam roller (1) to heat itself, without requiring a microwave oven for heating as needed for a prior art device.

In addition, the textured exterior surface (20) of the cordless foam roller (1) stimulates nerve endings more effectively and reaches deeper into the muscle's myofascial layers compared to a conventional soft foam roller having a smooth surface.

The cordless foam roller (1) has dimensions suitable for use with different muscles. In the various embodiments, the cordless foam roller (1) preferably has a length of about 18.5 inches and a diameter of about 4.5 inches. This preferred length enables the cordless foam roller (1) to be compact for storing in a gym bag or for use in a limited workout space, lightweight and portable for carrying to and from the gym, and useful for various large (e.g., back, shoulders) and small muscles (e.g., arms, calves) without risk of the user rolling off or lacking sufficient length to target a particular muscle. In addition, the preferred diameter allows the cordless foam roller (1) to be suitable for deeper, targeted massage.

Further, in the various embodiments, the cordless foam roller (1) is powered by a rechargeable battery, and thus can operate without power cords or cables attached to electrical outlets to provide mains power, allowing the foam roller (1) to be self-contained (i.e., having an internal or built-in power supply) and having greater mobility during use. In the various embodiments, the battery (38) comprises a lithium-ion battery which lasts about two the three years before replacement is needed.

### ADDITIONAL DISCLOSURES

The following are non-limiting, specific embodiments of the cordless foam roller and methods of facilitating a musculoskeletal rehabilitative, therapeutic or stretching program for a user:

Embodiment A. A cordless foam roller comprising: an elongated cylindrically-shaped body having a textured exterior surface, and defining an inner cavity for receiving and accommodating an inner console; the inner console defining an inner cavity housing one or more of a circuit board comprising a microcontroller and heat and vibration setting buttons; a battery; and a rotary motor; and having an outer surface supporting one or more heating elements thereon.

Embodiment B. The cordless foam roller of Embodiment A, further comprising a first end and a second end capped by a first end cap and a second end cap

Embodiment C. The cordless foam roller of any one of Embodiments A through B, wherein the textured exterior surface comprises a tread of chevrons, each chevron being disposed with a channel defined between adjacent chevrons.

Embodiment D. The cordless foam roller of any one of Embodiments A through C, wherein the body is formed of a thermally conductive material.

Embodiment E. The cordless foam roller of any one of Embodiments A through D, wherein the thermally conductive material is selected from silicone, a thermoplastic vulcanizate, or a thermal silicone compound doped with silver nanoparticles.

Embodiment F. The cordless foam roller of any one of Embodiments A through E, wherein the inner console comprises a pipe formed of a thermoplastic polymer.

Embodiment G. The cordless foam roller of any one of Embodiments A through F, wherein the thermoplastic polymer comprises acrylonitrile butadiene styrene.

Embodiment H. The cordless foam roller of any one of Embodiments A through G, wherein the microcontroller, the heat and vibration setting buttons, the battery, the rotary motor, and the one or more heating elements are operably connected to the circuit board.

Embodiment I. The cordless foam roller of any one of Embodiments A through H, wherein the microcontroller is configured to control power distribution to the battery and the rotary motor.

Embodiment J. The cordless foam roller of any one of Embodiments A through I, wherein the battery comprises a lithium-ion battery.

Embodiment K. The cordless foam roller of any one of Embodiments A through J, wherein the microcontroller is configured to transmit a signal to the rotary motor to operate at a programmed frequency selected by a user.

Embodiment L. The cordless foam roller of any one of Embodiments A through K, wherein the rotary motor is configured to spin at the programmed frequency along the longitudinal axis of the foam roller and to emit vibrations in a perpendicular direction relative to the longitudinal axis of the foam roller.

Embodiment M. The cordless foam roller of any one of Embodiments A through L, wherein the microcontroller is configured to transmit a signal to the battery to furnish electrical current sufficient for programmed heat selected by a user.

Embodiment N. The cordless foam roller of any one of Embodiments A through M, wherein the electrical current runs through the one or more heating elements for emitting heat, the heat being transferred through the body of the foam roller to the user.

Embodiment O. The cordless foam roller of any one of Embodiments A through N, wherein the amount of heat ranges from about 35° C. to about 55° C.

Embodiment P. The cordless foam roller of any one of Embodiments A through O, wherein the heat settings equate to temperatures of about 35° C., about 45° C., and about 55° C.

Embodiment Q. The cordless foam roller of any one of Embodiments A through P, further comprising at least one temperature sensor for detecting the temperature within the inner console and transmitting a signal to the microcontroller to increase or decrease the flow of the electrical current to the one or more heating elements.

Embodiment R. The cordless foam roller of any one of Embodiments A through Q, wherein the one or more heating elements comprises one or more heating pads mounted on an entirety or a portion of an outer surface of the inner console, and operably connected to the microcontroller.

Embodiment S. The cordless foam roller of any one of Embodiments A through Q, wherein the one or more heating elements comprises electrical resistive wires arranged in U-shapes along an entirety or portion of an outer surface of the inner console, and operably connected to the microcontroller.

Embodiment T. The cordless foam roller of any one of Embodiments A through S, wherein the microcontroller is configured to transmit a signal to activate a cooling fan to cool the temperature of the inner console.

Embodiment U. The cordless foam roller of any one of Embodiments A through T, wherein the microcontroller is configured to monitor and control supply voltage available from the battery, and shut down the battery.

Embodiment V. A method of facilitating a musculoskeletal rehabilitative, therapeutic or stretching program for a user, comprising the steps of: providing a cordless foam roller comprising an elongated cylindrically-shaped body having a textured exterior surface, and defining an inner cavity for receiving and accommodating an inner console; the inner console defining an inner cavity housing at least a circuit board comprising a microcontroller and heat and vibration setting buttons; a battery; and a rotary motor; and having an outer surface supporting one or more heating elements thereon; configuring the microcontroller to transmit a signal to the rotary motor to operate at a programmed frequency selected by the user, thereby spinning the rotary motor to emit vibration along the cordless foam roller; and configuring the microcontroller to transmit a signal to the battery to furnish electrical current sufficient for the programmed heat selected by the user, the electrical current running through the one or more heating elements to emit heat transferable through the body of the cordless foam roller to the user.

Embodiment W. The method of Embodiment V, further comprising the step of providing one or more sensors for detecting the temperature of the inner console.

Embodiment X. The method of any one of Embodiments V through W, further comprising the step of adjusting the temperature of the inner console by increasing or decreasing the flow of the electrical current to the one or more heating elements.

Additional embodiments which result from combining, integrating and/or omitting features of the embodiments explicitly described herein are not intended to be precluded. Although various embodiments have been shown and described, the invention is not limited to such embodiments and will be understood to include all modifications and variations as would be apparent to one skilled in the art. It should be understood that the invention is not intended to be limited to the particular forms disclosed. Rather, the intention is to cover all modifications, equivalents and alternatives falling within the scope of the invention as defined by the appended claims.

1. A cordless foam roller comprising:
an elongated cylindrically-shaped body having a textured exterior surface, and defining an inner cavity for receiving and accommodating an inner console;
the inner console defining an inner cavity housing one or more of a circuit board comprising a microcontroller and heat and vibration setting buttons; a battery; and a rotary motor; and having an outer surface supporting one or more heating elements thereon.

2. The cordless foam roller of claim 1, further comprising a first end and a second end capped by a first end cap and a second end cap.

3. The cordless foam roller of claim 1, wherein the textured exterior surface comprises a tread of chevrons, each chevron being disposed with a channel defined between adjacent chevrons.

4. The cordless foam roller of claim 1, wherein the body is formed of a thermally conductive material selected from silicone, a thermoplastic vulcanizate, a thermal silicone compound doped with silver nanoparticles, or other thermally conductive material.

5. (canceled)

6. The cordless foam roller of claim 1, wherein the inner console comprises a pipe formed of a thermoplastic polymer selected from acrylonitrile butadiene styrene or other thermoplastic polymer.

7. (canceled)

8. The cordless foam roller of claim 1, wherein the microcontroller, the heat and vibration setting buttons, the battery, the rotary motor, and the one or more heating elements are operably connected to the circuit board.

9. The cordless foam roller of claim 8, wherein the microcontroller is configured to control power distribution to the battery and the rotary motor.

10. The cordless foam roller of claim 8, wherein the battery comprises a lithium-ion battery.

11. The cordless foam roller of claim 9, wherein the microcontroller is configured to transmit a signal to the rotary motor to operate at a programmed frequency selected by a user.

12. The cordless foam roller of claim 11, wherein the rotary motor is configured to spin at the programmed frequency along the longitudinal axis of the foam roller and to emit vibrations in a perpendicular direction relative to the longitudinal axis of the foam roller.

13. The cordless foam roller of claim 8, wherein the microcontroller is configured to transmit a signal to the battery to furnish electrical current sufficient for programmed heat selected by a user.

14. The cordless foam roller of claim 13, wherein the electrical current runs through the one or more heating elements for emitting heat, the heat being transferred through the body of the foam roller to the user.

15. The cordless foam roller of claim 14, wherein the amount of heat ranges from about 35° C. to about 55° C., and the heat settings equate to temperatures of about 35° C., about 45° C., and about 55° C.

16. (canceled)

17. The cordless foam roller of claim 14, further comprising at least one temperature sensor for detecting the temperature within the inner console and transmitting a signal to the microcontroller to increase or decrease the flow of the electrical current to the one or more heating elements.

18. The cordless foam roller of claim 17, wherein the one or more heating elements comprises one or more heating pads mounted on an entirety or a portion of an outer surface of the inner console, and operably connected to the microcontroller.

19. The cordless foam roller of claim 17, wherein the one or more heating elements comprises electrical resistive wires arranged in U-shapes along an entirety or portion of an outer surface of the inner console, and operably connected to the microcontroller.

20. The cordless foam roller of claim 17, wherein the microcontroller is configured to transmit a signal to activate a cooling fan to cool the temperature of the inner console.

21. The cordless foam roller of claim 17, wherein the microcontroller is configured to monitor and control supply voltage available from the battery, and shut down the battery.

22. A method of facilitating a musculoskeletal rehabilitative, therapeutic or stretching program for a user, comprising the steps of:
providing a cordless foam roller comprising an elongated cylindrically-shaped body having a textured exterior surface, and defining an inner cavity for receiving and accommodating an inner console; the inner console defining an inner cavity housing at least a circuit board comprising a microcontroller and heat and vibration setting buttons; a battery; and a rotary motor; and having an outer surface supporting one or more heating elements thereon;
configuring the microcontroller to transmit a signal to the rotary motor to operate at a programmed frequency selected by the user, thereby spinning the rotary motor to emit vibration along the cordless foam roller; and
configuring the microcontroller to transmit a signal to the battery to furnish electrical current sufficient for the programmed heat selected by the user, the electrical current running through the one or more heating elements to emit heat transferable through the body of the cordless foam roller to the user.

23. The method of claim 22, further comprising the step of providing one or more sensors for detecting the temperature of the inner console, and adjusting the temperature of the inner console by increasing or decreasing the flow of the electrical current to the one or more heating elements.
